(19) United States
(12) Patent Application Publication
(10) Pub. No.: US 2019/0309030 A1
(43) Pub. Date: Oct. 10, 2019

(54) ACRIDINE LABELLED CONJUGATES AND PREPARATION METHODS THEREFOR AND CHEMILUMINESCENT KITS

(71) Applicant: SHENZHEN YHLO BIOTECH CO., LTD., SHENZHEN (CN)

(72) Inventors: Fuzhen XIA, SHENZHEN (CN); Taoxu LIU, SHENZHEN (CN); Liang ZHU, SHENZHEN (CN); Chengyong XIAO, SHENZHEN (CN); Chungen QIAN, SHENZHEN (CN)

(21) Appl. No.: 16/315,167

(22) PCT Filed: Jul. 5, 2016
(86) PCT No.: PCT/CN2016/088568

§ 371 Date: Jan. 4, 2019

(51) Int. Cl. C07K 14/435; C07K 16/26; C07K 1/13; G01N 33/68; G01N 21/76

(52) U.S. Cl. CPC C07K 14/435; C07K 16/26; G01N 21/76; G01N 33/6803; G01N 33/6854; C07K 1/13

(57) ABSTRACT

Disclosed are an acridine marker conjugate and a preparation method therefor and a chemiluminescent kit. The acridine marker conjugate comprises an acridine substitute, a carrier protein and a protein to be marked sequentially connected. The carrier protein is a protein containing carboxyl and amino, a modified protein, a polypeptide or a modified polypeptide; and the protein to be marked is a protein containing amino, a modified protein, a polypeptide or a modified polypeptide.

### FIELD

The present disclosure relates to the field of in vitro detection, in particular to an acridine-labelled conjugate, a method for preparing the same and a chemiluminescent kit.

### BACKGROUND

Chemiluminescent labeling immunoassay, also known as chemiluminescence immunoassay (CLIA), is an immunoassay in which an antigen, a hapten, or an antibody is directly labeled with a chemiluminescent agent. A chemiluminescent substance used for labeling includes substituted acridine, and depending on different substituents, the chemiluminescent substance is divided into two categories: acridinium ester (AE) and acridine sulfonamide, both of which are effective luminescent labels and under action of a start luminescent reagent (NaOH, H₂O₂), generates a luminescence, which is an intense direct luminescence that completed in one second and is a fast blinking luminescence.

Substituted acridine, when is used in immunoassay as a chemiluminescent label, allows a simple and rapid chemical reaction without a catalyst and detection of a small molecule antigen by competition and a macromolecule antigen by sandwich assay, and has low non-specific binding and background. Substituted acridine will not bring a reduced amount of luminescence generated when binding to a macromolecule, and thus sensitivity improves. In terms of luminescence mechanism, such compounds are characterized by: 1. during a luminescent reaction, before formation of an electronically excited intermediate, a non-luminescent substituent moiety bound to a acridine ring is detached from the acridine ring, i.e., a non-luminescent moiety is separated from a luminescent moiety, and thus a structure of substituent substantially has no effect on luminous efficiency of substituted acridine. 2. No catalyst is required for chemiluminescence of acridinium ester or acridine sulfonamide compounds, and such compounds are capable of luminescence in a dilute alkaline solution containing H₂O₂. Therefore, when applied in chemiluminescence assay, acridinium ester or acridine sulfonamide compounds have many advantages, mainly comprising: \textcircled{1} low background luminescence and high signal to noise ratio; \textcircled{2} less interference factors for luminescence reaction; \textcircled{3} fast and focused release of light, high luminescent efficiency and luminescent intensity; \textcircled{4} prone to bind to a protein without a decreased photon yield after binding; and \textcircled{5} stable labels (which can be stored for several months at 2° C. to 8° C.). Substituted acridine is therefore a very effective and a very good chemiluminescent label.

An acridine-labelled conjugate is a complex obtained by binding substituted acridine to a substance to be labelled (antibody, antigen, and the like). The acridine-labelled conjugate has a quality that is directly related to whether the chemiluminescent immunoassay succeeds or not, and thus is referred to as a key reagent.

So far, carbodiimide crosslinking is used as a conventional method for preparing the acridine-labelled conjugate, wherein a carbodiimide crosslinking agent is used as a bridge for binding substituted acridine to a protein to be labelled. However, in the acridine-labelled conjugate prepared by the conventional method, substituted acridine is bound to the protein to be labelled by carbodiimide, and frequently interferes with an active site on the protein to be labelled, resulting in decreased activity of the acridine-labelled conjugate, and affecting sensitivity for the immunoassay.

### SUMMARY

Based on this, it is necessary to provide an acridine-labelled conjugate with relatively high activity, a method for preparing the same, and a chemiluminescent kit.

An acridine-labelled conjugate comprises a substituted acridine, a carrier protein, and a protein to be labelled which are sequentially linked;

the carrier protein is a protein, a modified protein, a polypeptide or a modified polypeptide containing a carboxyl group and an amino group, and the carrier protein reacts, by the amino group therein, with the substituted acridine to form a chemical bond;

the protein to be labelled is a protein, a modified protein, a polypeptide or a modified polypeptide containing an amino group, and the amino group in the protein to be labelled reacts with the carboxyl group in the carrier protein to form a —NH—CO— structure, thereby linking the carrier protein and the protein to be labelled together.

A method for preparing the acridine-labelled conjugate described above, comprises:

covalently cross-linking and fully reacting a substituted acridine with a carrier protein to obtain a substituted acridine-carrier protein conjugate, wherein the carrier protein is a protein, a modified protein, a polypeptide or a modified polypeptide containing a carboxyl group and an amino group, and the carrier protein reacts, by the amino group therein, with the substituted acridine to form a chemical bond;

purifying the substituted acridine-carrier protein conjugate;

activating the carboxyl group in a purified substituted acridine-carrier protein conjugate by using a crosslinking agent; and

cross-linking and fully reacting a carboxyl group-activated substituted acridine-carrier protein conjugate with a protein to be labelled to obtain an acridine-labelled conjugate, wherein the acridine-labelled conjugate comprises the substituted acridine, the carrier protein and the protein to be labelled which are sequentially linked, the protein to be labelled is a protein, a modified protein, a polypeptide or a modified polypeptide containing an amino group, and the amino group in the protein to be labelled reacts with the carboxyl group in the carrier protein to form a —NH—CO— structure, thereby linking the carrier protein and the protein to be labelled together.

A chemiluminescence kit for binding a protein to be labelled to form the acridine-labelled conjugate described as above, comprises a substituted acridine and a carrier protein;

the carrier protein is a protein, a modified protein, a polypeptide or a modified polypeptide containing a carboxyl group and an amino group, and the carrier protein may reacts, by the amino group therein, with the substituted acridine to form a chemical bond;

the protein to be labelled is a protein, a modified protein, a polypeptide or a modified polypeptide containing an amino group, and the amino group in the protein to be labelled may react with the carboxyl group in the carrier protein to form a —NH—CO— structure, thereby linking the carrier protein and the protein to be labelled together.

Such an acridine-labelled conjugate comprises a substituted acridine, a carrier protein, and a protein to be labelled which are sequentially linked, the carrier protein reacts, by an amino group therein, with the substituted acridine to form a chemical bond, and the amino group in the protein to be labelled reacts with the carboxyl group in the carrier protein to form a —NH—CO— structure, thereby linking the carrier protein and the protein to be labelled together. Due to a relatively identified binding site, the substituted acridine is prevented from interfering with an active site in the protein to be labelled, and thus the acridine-labelled conjugate has relatively high activity. In addition, the carrier protein allows an increased steric hindrance of the acridine-labelled conjugate, thereby increasing the sensitivity when using the acridine-labelled conjugate.

### BRIEF DESCRIPTION OF THE DRAWINGS

FIG. 1 is a flowchart of a method for preparing an acridine-labelled conjugate according to an embodiment.

FIG. 2 is a scatter gram for results from detecting TSH antigen samples having a serial of concentrations in a test example.

FIG. 3 is a scatter gram for results from detecting E2 antigen samples having a serial of concentrations in a test example.

### DETAILED DESCRIPTION OF THE INVENTION

Specific embodiments of the present disclosure will be described in detail below with reference to drawings and specific examples. Numerous specific details are set forth in the description below in order to provide a thorough understanding of the disclosure. However, the present disclosure can be implemented in many other ways than those described herein, and those skilled in the art may make similar modifications without departing from the scope of the present disclosure, and thus the present disclosure is not limited by the specific embodiments disclosed below.

An acridine-labelled conjugate comprises a substituted acridine, a carrier protein, and a protein to be labelled which are sequentially linked.

The carrier protein is a protein, a modified protein, a polypeptide or a modified polypeptide containing a carboxyl group and an amino group, and reacts, by an amino group in the carrier protein, with the substituted acridine to form a chemical bond.

The carrier protein may be a protein or a polypeptide having a carboxyl group and an amino group per se, or may be a modified protein or a modified polypeptide into which a carboxyl group and an amino group are introduced by modification.

In this embodiment, the carrier protein may be bovine serum albumin, chicken serum albumin or hemocyanin.

The protein to be labelled is a protein, a modified protein, a polypeptide or a modified polypeptide containing an amino group, and the amino group in the protein to be labelled reacts with the carboxyl group in the carrier protein to form a —NH—CO— structure, thereby linking the carrier protein and the protein to be labelled together.

The protein to be labelled may be a protein or a polypeptide having an amino group per se, or may be a modified protein or a modified polypeptide into which an amino group is introduced by modification.

In this embodiment, the protein to be labelled is an antigen, a hapten or an antibody.

The substituted acridine may be acridinium ester (DMAE-NHS, AE-NHS), acridinic acid (acridine-9-carboxylic acid hydrate), acridine amide, or acridine sulfonamide (NSP-SA-NHS). Depending on different specific substituted acridines, the amino group in the carrier protein reacts with a different group (carboxyl, succinimidyl ester, and the like) in the substituted acridine to form a chemical bond.

In particular, acridinium ester may be AE-NHS (10-methyl-acridine-9-N-succinimidyl ester formic ester), DMAE-NHS (2′,6′-dimethyl-4′-(N-succinimidyloxycarbonyl)phenyl-acridine-9-formate), ME-DMAE-NHS (2′,6′-dimethylcarbonylphenyl-10-methyl-9-acridinecarboxylate-4′-N-succinimidyl ester-trifluoromethanesulfonate), and the like.

For example, if acridinium ester is AE-NHS, the acridine-labelled conjugate has following structural formula:

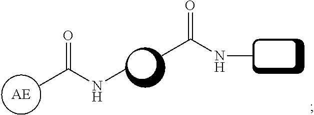

wherein,

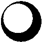

is the carrier protein having an amino residue and a carboxyl residue, and

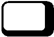

is the protein to be labeled having an amino residue.

Such an acridine-labelled conjugate comprises a substituted acridine, a carrier protein, and a protein to be labelled which are sequentially linked, the carrier protein reacts, by an amino group therein, with the substituted acridine to form a chemical bond, and the amino group in the protein to be labelled reacts with the carboxyl group in the carrier protein to form a —NH—CO— structure, thereby linking the carrier protein and the protein to be labelled together. Due to a relatively identified binding site, the substituted acridine is prevented from interfering with an active site in the protein to be labelled, and thus the acridine-labelled conjugate has relatively high activity. In addition, the carrier protein allows an increased steric hindrance of the acridine-labelled conjugate, thereby increasing sensitivity when using the acridine-labelled conjugate.

In addition, the carrier protein allows an increased steric hindrance of the acridine-labelled conjugate, thereby increasing sensitivity when using the acridine-labelled conjugate.

Compared with the conventional technology, in such an acridine-labelled conjugate containing the carrier protein, the protein to be labelled is prevented from inactivation due to an effectively protected active site thereof, and the acridine-labelled conjugate is characterized by high stability, controllable amount of linkage, and the like by sequentially linking the substituted acridine, the carrier protein and the label to be labelled via the chemical bond.

Such acridine-labelled conjugate can be directly used for detection and quantitative analysis in a chemiluminescence immunoassay, and can solve disadvantages of acridine-labelled conjugate prepared by conventional carbodiimide cross-linking method and the like, such as inactivation of a start material, poor signal and the like.

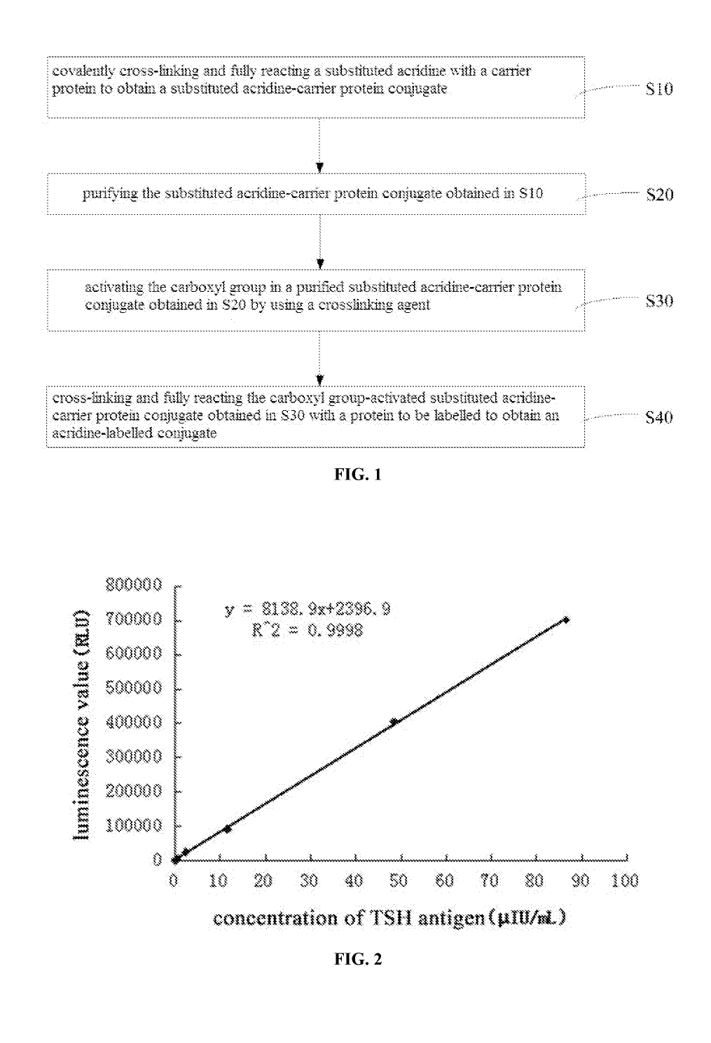

A method for preparing the acridine-labelled conjugate described above as shown in FIG. 1 comprises:

S10, covalently cross-linking and fully reacting a substituted acridine with a carrier protein to obtain a substituted acridine-carrier protein conjugate.

In covalently crosslinking the substituted acridine with the carrier protein, a molar ratio of the substituted acridine to the carrier protein ranges from 100:1 to 20000:1.

Preferably, the molar ratio of the substituted acridine to the carrier protein ranges from 500:1 to 5000:1.

The carrier protein is a protein, a modified protein, a polypeptide or a modified polypeptide containing a carboxyl group and an amino group, and the carrier protein reacts, by the amino group therein, with the substituted acridine to form a chemical bond.

The carrier protein may be a protein or a polypeptide having a carboxyl group and an amino group per se, or may be a modified protein or a modified polypeptide into which a carboxyl group and an amino group are introduced by modification.

In this embodiment, the carrier protein may be bovine serum albumin, chicken serum albumin or hemocyanin.

The substituted acridine may be acridinium ester (DMAE-NHS, AE-NHS), acridinic acid (acridine-9-carboxylic acid hydrate), acridine amide or acridine sulfonamide (NSP-SA-NHS). Depending on different specific substituted acridines, the amino group in the carrier protein reacts with a different group (carboxyl, succinimidyl ester, and the like) in the substituted acridine to form a chemical bond.

In particular, acridinium ester may be AE-NHS (10-methyl-acridine-9-N-succinimidyl ester formic ester), DMAE-NHS (2′,6′-dimethyl-4′-(N-succinimidyloxycarbonyl)phenyl-acridine-9-formic ester), ME-DMAE-NHS (2′,6′-dimethylcarbonylphenyl-10-methyl-9-acridinecarboxylate-4′-N-succinimidyl ester-trifluoromethanesulfonate), and the like.

For example, if acridinium ester is AE-NHS, the substituted acridine is covalently cross-linked with the carrier protein to obtain a substituted acridine-carrier protein conjugate, as shown in following reaction scheme:

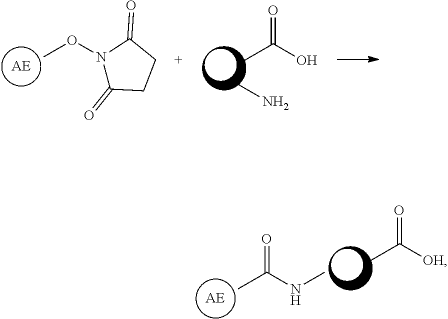

wherein

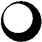

is the carrier protein having an amino residue and a carboxyl residue, and AE-NHS is linked to the carrier protein via an amide bond.

Specific reaction process for the above reaction scheme is as follows: adding and fully dissolving the carrier protein in a buffer before adding excess acridinium ester, reacting at a temperature ranging from 4° C. to 37° C. for 0.5 h to 12 h, and after completion of the reaction, purifying to obtain an acridinium ester-carrier protein conjugate, wherein the buffer is phosphate buffered saline, carbonate buffered saline, 2-(N-morpholino)ethanesulfonic acid (MES) buffer or piperazine-N,N′-bis(2-ethanesulfonic acid) (PIPES) buffer, and the reaction is performed at a pH ranging from 4 to 10.

Since the acridinium ester is present at an excess amount with respective to the carrier protein, the amino group in the carrier protein may be regarded as being completely exhausted and will not compete with the amino group in the protein to be labelled in a next stage, and thus no amino group in the acridinium ester-carrier protein conjugate needs to be blocked.

S20, purifying the substituted acridine-carrier protein conjugate obtained in S10.

Purifying the substituted acridine-carrier protein conjugate may be one or more selected from the group consisting of ultrafiltration purification, desalting column purification, and dialysis purification.

S30, activating the carboxyl group in a purified substituted acridine-carrier protein conjugate obtained in S20 by using a crosslinking agent.

The crosslinking agent comprises carbodiimide and hydroxysuccinimide, and a molar ratio of carbodiimide to the substituted acridine-carrier protein conjugate ranges from 10:1 to 5000:1, and a molar ratio of carbodiimide to hydroxysuccinimide ranges from 5:1 to 1:10.

Preferably, carbodiimide is at least one selected from dicyclohexylcarbodiimide, 1-(3-dimethyl aminopropyl)-3-ethyl carbodiimide, and N,N′-diisopropyl carbodiimide.

Preferably, the molar ratio of carbodiimide to the substituted acridine-carrier protein conjugate ranges from 50:1 to 1000:1.

Preferably, hydroxysuccinimide is at least one selected from N-hydroxysuccinimide and N-hydroxysulfosuccinimide.

Preferably, the molar ratio of carbodiimide to hydroxysuccinimide ranges from 2:1 to 1:5.

Preferably, S30 further comprises: adding mercaptoethanol for quenching activity of the cross-linking agent (or purifying for removal of the cross-linking agent) after the carboxyl group in the substituted acridine-carrier protein conjugate is activated, so as to obtain a carboxyl group-activated substituted acridine-carrier protein conjugate having a blocked amino group.

S40, cross-linking and fully reacting the carboxyl group-activated substituted acridine-carrier protein conjugate obtained in S30 with a protein to be labelled to obtain an acridine-labelled conjugate.

The resulting acridine-labelled conjugate comprises the substituted acridine, the carrier protein and the protein to be labelled which are sequentially linked.

The protein to be labelled is a protein, a modified protein, a polypeptide or a modified polypeptide containing an amino group, and the amino group in the protein to be labelled reacts with the carboxyl group in the carrier protein to form a —NH—CO— structure, thereby linking the carrier protein and the protein to be labelled together.

The protein to be labelled may be a protein or a polypeptide having an amino group per se, or may be a modified protein or a modified polypeptide into which an amino group is introduced by modification.

In this embodiment, the protein to be labelled is an antigen, a hapten or an antibody.

In cross-linking the carboxyl group-activated substituted acridine-carrier protein conjugate with the protein to be labelled, a molar ratio of the substituted acridine-carrier protein conjugate to the protein to be labelled ranges from 5:1 to 1:5.

Preferably, the molar ratio of the substituted acridine-carrier protein conjugate to the protein to be labelled ranges from 2:1 to 1:2.

For example, if acridinium ester is AE-NHS, an carboxyl group of substituted acridine-carrier protein conjugate is activated by using carbodiimide and hydroxysuccinimide, and the carboxyl group-activated substituted acridine-carrier protein conjugate is cross-linked with the protein to be labelled to obtain an acridine-labelled conjugate, as shown in following reaction scheme:

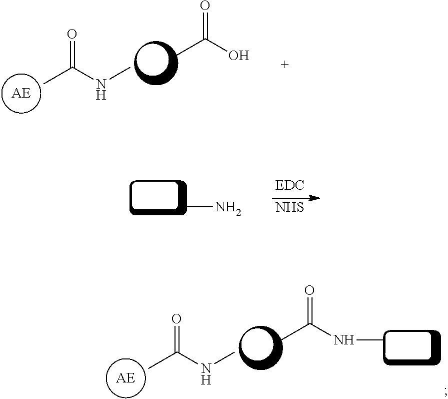

wherein,

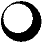

is the carrier protein having an amino residue and a carboxyl residue, and

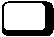

is the protein to be labelled having an amino residue.

Specific reaction process of the above reaction scheme is as follows: adding and fully dissolving the substituted acridine-carrier protein conjugate in a buffer; adding EDC and NHS and reacting at 25° C. for 10 min; then adding the protein to be labelled and reacting at a temperature ranging from 4° C. to 37° C. for 0.5 h to 12 h; and after completion of the reaction, purifying to obtain an acridine-labelled conjugate, wherein the buffer is phosphate buffered saline, carbonate buffered saline, 2-(N-morpholino)ethanesulfonic acid (MES) buffer or piperazine-N,N′-bis(2-ethanesulfonic acid) (PIPES) buffer, and the reaction is performed at a pH ranging from 4 to 10.

In such a method for preparing the acridine-labelled conjugate, the carrier protein reacts, by an amino group therein, with the substituted acridine to form a chemical bond, and the amino group in the protein to be labelled reacts with the carboxyl group in the carrier protein to form a —NH—CO— structure, thereby linking the carrier protein and the protein to be labelled together. Due to a relatively identified binding site, due to a relatively identified binding site, the substituted acridine is prevented from interfering with an active site in the protein to be labelled, and thus the acridine-labelled conjugate produced by such method for preparing the acridine-labelled conjugate has relatively high activity.

In addition, the carrier protein allows an increased steric hindrance of the acridine-labelled conjugate, thereby increasing sensitivity when using the acridine-labelled conjugate.

The present disclosure also discloses a chemiluminescence kit for binding a protein to be labelled to form the acridine-labelled conjugate described above.

The chemiluminescence kit comprises a substituted acridine and a carrier protein.

The substituted acridine may be acridinium ester (DMAE-NHS, AE-NHS), acridinic acid (acridine-9-carboxylic acid hydrate), acridine amide or acridine sulfonamide (NSP-SA-NHS). Depending on different specific substituted acridines, the amino group in the carrier protein reacts with a different group (carboxyl, succinimidyl ester, and the like) in the substituted acridine to form a chemical bond.

In particular, the acridinium ester may be AE-NHS (10-methyl-acridine-9-N-succinimidyl ester formic ester), DMAE-NHS (2′,6′-dimethyl-4′-(N-succinimidyloxycarbonyl)phenyl-acridine-9-formic ester), ME-DMAE-NHS (2′,6′-dimethylcarbonylphenyl-10-methyl-9-acridinecarboxylate-4′-N-succinimidyl ester-trifluoromethanesulfonate), and the like.

The carrier protein is a protein, a modified protein, a polypeptide or a modified polypeptide containing a carboxyl group and an amino group, and the carrier protein reacts, by an amino group therein, with the substituted acridine to form a chemical bond.

The carrier protein may be a protein or a polypeptide having a carboxyl group and an amino group per se, or may be a modified protein or a modified polypeptide into which a carboxyl group and an amino group are introduced by modification.

In this embodiment, the carrier protein may be bovine serum albumin, chicken serum albumin or hemocyanin.

The protein to be labelled is a protein, a modified protein, a polypeptide or a modified polypeptide containing an amino group, and the amino group in the protein to be labelled reacts with the carboxyl group on the carrier protein to form a —NH—CO— structure, thereby linking the carrier protein and the protein to be labelled together.

The protein to be labelled may be a protein or a polypeptide having an amino group per se, or may be a modified protein or a modified polypeptide into which an amino group is introduced by modification.

In this embodiment, the protein to be labelled is an antigen, a hapten or an antibody.

Preferably, the chemiluminescent kit further comprises at least one of a centrifugal desalting column and a centrifugal ultrafiltration tube.

In such a chemiluminescent kit, the substituted acridine, the carrier protein, and the protein to be labelled may be sequentially linked by chemical bonds. Due to a relatively identified binding site, the substituted acridine is prevented from interfering with an active site in the protein to be labelled, and thus the acridine-labelled conjugate formed has relatively high activity.

In addition, the carrier protein allows an increased steric hindrance of the acridine-labelled conjugate, thereby increasing sensitivity when using the acridine-labelled conjugate.

The following are specific examples.

### Example 1

1 mg of BSA was dissolved in 1 mL of 150 mM PBS buffer (pH 7.4), and 40 μL of 10 mg/mL acridinium ester in DMF was added. The mixture was reacted at 25° C. for 4 hrs and subjected to 5 mL desalting column (7 KD MWCO, Thermo fish) chromatography three times using 150 mM PBS (pH 7.4) as an elution buffer, to remove free acridinium ester and a by-product from the reaction and to obtain an acridine-BSA solution.

To the above purified acridine-BSA solution, were added EDC at a final concentration of 10 mmol/L and NHS at a final concentration of 20 mmol/L. After reaction at 25° C. for 10 min, TSH antibody was added (Santa cruz biotechnology, Cat. No.: sc-418393) was added, and thoroughly mixed. The mixture was reacted at 25° C. for 4 hrs and subjected to 5 mL desalting column (7 KD MWCO, Thermo fish) chromatography three times using 150 mM PBS (pH 7.4) as an elution buffer to remove free EDC and NHS and a by-product from the reaction and to obtain a solution of acridinium ester-labelled BSA-binding TSH monoclonal antibody.

### Example 2

1 mg of BSA was dissolved in 1 mL of 150 mM PBS buffer (pH 7.4), and 40 μL of 10 mg/mL acridinium ester in DMF was added. The mixture was reacted at 25° C. for 4 hrs and subjected to 5 mL desalting column (7 KD MWCO, Thermo fish) chromatography three times using 150 mM PBS (pH 7.4) as an elution buffer, to remove free acridinium ester and a by-product from the reaction and to obtain an acridine-BSA solution.

To the above purified acridine-BSA solution, were added EDC at a final concentration of 10 mmol/L and NHS at a final concentration of 20 mmol/L. After reaction at 25° C. for 10 min, estradiol antigen was added (abcam, Cat. No.: ab120657) was added, and thoroughly mixed. The mixture was reacted at 25° C. for 4 hrs and subjected to 5 mL desalting column (7 KD MWCO, Thermo fish) chromatography three times using 150 mM PBS (pH 7.4) as an elution buffer to remove free EDC and NHS and a by-product from the reaction and to obtain a solution of acridinium ester-labelled BSA-binding estradiol antigen.

### Example 3

1 mg of OVA was dissolved in 1 mL of 150 mM PBS buffer (pH 7.4), and 60 μL of 10 mg/mL acridinium ester in DMF was added. The mixture was reacted at 25° C. for 4 hrs and subjected to 5 mL desalting column (7 KD MWCO, Thermo fish) chromatography three times using 150 mM PBS (pH 7.4) as an elution buffer, to remove free acridinium ester and a by-product from the reaction and to obtain an acridine-OVA solution.

To the above purified acridine-OVA solution, were added EDC at a final concentration of 10 mmol/L and NHS at a final concentration of 20 mmol/L. After reaction at 25° C. for 10 min, TSH antibody was added (Santa cruz biotechnology, Cat. No.: sc-418393) was added, and thoroughly mixed. The mixture was reacted at 25° C. for 4 hrs and subjected to 5 mL desalting column (7 KD MWCO, Thermo fish) chromatography three times using 150 mM PBS (pH 7.4) as an elution buffer to remove free EDC and NHS and a by-product from the reaction and to obtain a solution of acridinium ester-labelled OVA-binding TSH monoclonal antibody.

### Example 4

1 mg of OVA was dissolved in 1 mL of 150 mM PBS buffer (pH 7.4), and 60 μL of 10 mg/mL acridinium ester in DMF was added. The mixture was reacted at 25° C. for 4 hrs and subjected to 5 mL desalting column (7 KD MWCO, Thermo fish) chromatography three times using 150 mM PBS (pH 7.4) as an elution buffer, to remove free acridinium ester and a by-product from the reaction and to obtain an acridine-OVA solution.

To the above purified acridine-OVA solution, were added EDC at a final concentration of 10 mmol/L and NHS at a final concentration of 20 mmol/L. After reaction at 25° C. for 10 min, estradiol antigen was added (abcam, Cat. No.: ab120657) was added, and thoroughly mixed. The mixture was reacted at 25° C. for 4 hrs and subjected to 5 mL desalting column (7 KD MWCO, Thermo fish) chromatography three times using 150 mM PBS (pH 7.4) as an elution buffer to remove free EDC and NHS and a by-product from the reaction and to obtain a solution of acridinium ester-labelled OVA-binding estradiol antigen.

### Example 5

1 mg of KLH was dissolved in 1 mL of 150 mM PBS buffer (pH 7.4), and 5 μL of 10 mg/mL acridinium ester in DMF was added. The mixture was reacted at 25° C. for 4 hrs and subjected to 5 mL desalting column (7 KD MWCO, Thermo fish) chromatography three times using 150 mM PBS (pH 7.4) as an elution buffer, to remove free acridinium ester and a by-product from the reaction and to obtain an acridine-KLH solution.

To the above purified acridine-KLH solution, were added EDC at a final concentration of 10 mmol/L and NHS at a final concentration of 20 mmol/L. After reaction at 25° C. for 10 min, TSH antibody was added (Santa cruz biotechnology, Cat. No.: sc-418393) was added, and thoroughly mixed. The mixture was reacted at 25° C. for 4 hrs and subjected to 5 mL desalting column (7 KD MWCO, Thermo fish) chromatography three times using 150 mM PBS (pH 7.4) as an elution buffer to remove free EDC and NHS and a by-product from the reaction and to obtain a solution of acridinium ester-labelled KLH-binding TSH monoclonal antibody.

### Example 6

1 mg of KLH was dissolved in 1 mL of 150 mM PBS buffer (pH 7.4), and 5 μL of 10 mg/mL acridinium ester in DMF was added. The mixture was reacted at 25° C. for 4 hrs and subjected to 5 mL desalting column (7 KD MWCO, Thermo fish) chromatography three times using 150 mM PBS (pH 7.4) as an elution buffer, to remove free acridinium ester and a by-product from the reaction and to obtain an acridine-KLH solution.

To the above purified acridine-KLH solution, were added EDC at a final concentration of 10 mmol/L and NHS at a final concentration of 20 mmol/L. After reaction at 25° C. for 10 min, estradiol antigen was added (abcam, Cat. No.: ab120657) was added, and thoroughly mixed. The mixture was reacted at 25° C. for 4 hrs and subjected to 5 mL desalting column (7 KD MWCO, Thermo fish) chromatography three times using 150 mM PBS (pH 7.4) as an elution buffer to remove free EDC and NHS and a by-product from the reaction and to obtain a solution of acridinium ester-labelled KLH-binding estradiol antigen.

### Comparative Example 1

1 mg of TSH antibody (Santa cruz biotechnology, Cat. No.: sc-418393) was dissolved in 1 mL of 150 mM PBS buffer (pH 7.4), and EDC (at a final concentration of 10 mmol/L) and NHS (at a final concentration of 20 mmol/L) were added. After reaction at 25° C. for 10 min, 16 μL of 10 mg/mL acridinium ester in DMF was added and mixed thoroughly. The mixture was reacted at 25° C. for 4 hrs and subjected to 5 mL desalting column (7 KD MWCO, Thermo fish) chromatography three times using 150 mM PBS (pH 7.4) as an elution buffer to remove free EDC, and NHS and a by-product from the reaction and to obtain a solution of acridinium ester-labelled TSH monoclonal antibody.

### Comparative Example 2

1 mg of estradiol antigen (abcam, Cat. No.: ab120657) was dissolved in 1 mL of 150 mM PBS buffer (pH 7.4), and EDC (at a final concentration of 10 mmol/L) and NHS (at a final concentration of 20 mmol/L) were added. After reaction at 25° C. for 10 min, 404, of 10 mg/mL acridinium ester in DMF was added and mixed thoroughly. The mixture was reacted at 25° C. for 4 hrs and subjected to 5 mL desalting column (7 KD MWCO, Thermo fish) chromatography three times using 150 mM PBS (pH 7.4) as an elution buffer to remove free EDC, and NHS and a by-product from the reaction and to obtain a solution of acridinium ester-labelled estradiol antigen.

### Test Example

The solution of acridinium ester-labelled carrier protein-binding anti-TSH antibody obtained in Examples 1, 3, 5 and the solution of acridinium ester-labelled anti-TSH antibody prepared by carbodiimide method in Comparative Example 1 were used in a chemiluminescence immunoassay, respectively.

To 20 μIU/mL of TSH sample, was added 20 μg of TSH monoclonal antibody-coated magnetic beads, respectively followed by 40 ng/mL acridinium ester-labelled anti-TSH antibody prepared by the various methods. The resulting solution was measured for a luminescence value using a full-automatic chemiluminescence immunoassay analyzer (SHENZHEN YHLO BIOTECH CO., LTD Type: iFlash 3000). Measurements were performed in triplicate, and an average value from the measurements was taken as results shown in Table 1. A greater luminescence value from the results in the immunoassay shows the acridine label has a better activity.

TABLE 1
Comparison between results from TSH
detection using acridine labels
prepared by the various methods

| | Average luminescence |
|---|---|
| acridine label | value (RLU) |
| Example 1 | 2327580 |
| Example 3 | 1882410 |
| Example 5 | 2032640 |
| Comparative Example 1 | 6010 |

From the results in Table 1, it can be seen that the acridine label reagents prepared in Examples 1, 3 and 5 exhibited significantly better activity than that prepared in Comparative Example 1.

The solution of acridine-labelled anti-TSH goat polyclonal antibody obtained in Example 1 was used for detecting TSH having a serial of concentrations in chemiluminescence immunoassay. To respective TSH sample solutions having a serial of concentrations, 20 μg of TSH monoclonal antibody-coated magnetic bead reagent, respectively followed by 40 ng/mL acridinium ester-labelled anti-TSH antibody prepared by the various methods. The resulting solution was measured for a luminescence value using a full-automatic chemiluminescence immunoassay analyzer (SHENZHEN YHLO BIOTECH CO., LTD., Type: iFlash 3000). Measurements were performed in triplicate, and an average value from the measurements was taken as results shown in Table 2, and data from Table 2 is plotted with the concentration of TSH antigen on the X-axis against the relative luminescence value plotted on the Y-axis, as shown in FIG. 2.

TABLE 2
Immunoassay results for TSH antigen samples having a
serial of concentrations

| TSH antigen concentration | Average Luminescence |
|---|---|
| (μIU/mL) | value (RLU) |
| 0.02 | 698 |
| 0.18 | 2967 |
| 0.39 | 4847 |
| 2.28 | 25098 |
| 11.55 | 92288 |
| 48.44 | 403477 |
| 86.48 | 702871 |

From the results in Table 2 and FIG. 2, it can be known that the acridine-label reagent prepared in Example 1 can be applied in chemiluminescence immunoassay and has a good effect. The method for preparing the acridinium ester label according to Example 1 has good applicability.

The solution of acridinium ester-labelled carrier protein-binding estradiol antigen obtained in Examples 2, 4, 6 and the solution of acridinium ester-labelled estradiol antigen prepared by carbodiimide method in Comparative Example 2 were used in a chemiluminescence immunoassay, respectively.

To 500 pg/mL of estradiol sample, was added 40 μg of estradiol monoclonal antibody-coated magnetic beads, respectively followed by a solution of 40 ng/mL acridinium ester-labelled estradiol antigen prepared by the various methods. The resulting solution was measured for a luminescence value using a full-automatic chemiluminescence immunoassay analyzer (SHENZHEN YHLO BIOTECH CO., LTD., Type: iFlash 3000). Measurements were performed in triplicate, and an average value from the measurements was taken as results shown in Table 3. A lower luminescence value from the results in the immunoassay shows the acridine label has a better activity.

TABLE 3
Comparison between results from E2 detection using acridine
labels prepared by the various methods

| | Average luminescence |
|---|---|
| acridine label | value (RLU) |
| Example 2 | 94734 |
| Example 4 | 103267 |
| Example 6 | 98247 |
| Comparative Example 2 | 643026 |

From the results in Table 3, it can be seen that the acridine label reagents prepared in Examples 2, 4 and 6 exhibited significantly better activity than that prepared in Comparative Example 2 and having almost no signal.

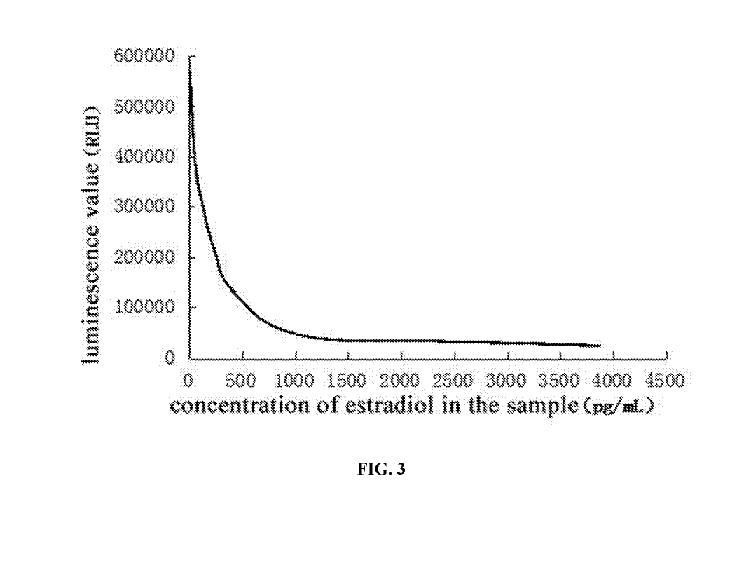

The solution of acridine-labelled carrier protein-binding estradiol antigen obtained in Example 2 was used for detecting E2 having a serial of concentrations in chemiluminescence immunoassay. To respective E2 sample solutions having a serial of concentrations, 40 μg of estradiol monoclonal antibody-coated magnetic bead reagent, respectively followed by 40 ng/mL acridinium ester-labelled anti-TSH antibody prepared by the various methods. The resulting solution was measured for a luminescence value using a full-automatic chemiluminescence immunoassay analyzer (SHENZHEN YHLO BIOTECH CO., LTD., Type: iFlash 3000). Measurements were performed in triplicate, and an average value from the measurements was taken as results shown in Table 4, and data from Table 4 is plotted with concentration of E2 antigen in the sample on the X-axis against the relative luminescence value plotted on the Y-axis, as shown in FIG. 3.

TABLE 4
Immunoassay results for E2 antigen samples
having a serial of concentrations

| E2 antigen concentration | Average Luminescence |
|---|---|
| (pg/mL) | value (RLU) |
| 2.4 | 571030 |
| 53.4 | 391438 |
| 131.7 | 298163 |
| 238.1 | 216681 |
| 417.0 | 134729 |
| 1021.4 | 48550 |
| 2388.9 | 35095 |
| 2958.0 | 30747 |
| 3872.5 | 25506 |

From the results in Table 4 and FIG. 7, it can be known that the acridine label reagent prepared in Example 2 can be applied in chemiluminescence immunoassay and has a good effect. The method for preparing the acridinium ester label according to Example 2 has good applicability.

The above-mentioned examples are merely illustrative of one or more embodiments of the present disclosure, and the description thereof is more specific and detailed, but should not to be construed as limiting the scope of the disclosure. It should be noted that various variations and modifications may be made by those skilled in the art without departing from the spirit and scope of the disclosure. Therefore, the scope of the disclosure should be subject to the appended claims.

1. An acridine-labelled conjugate, comprising:
a substituted acridine, a carrier protein, and a protein to be labelled which are sequentially linked;
the carrier protein is a protein, a modified protein, a polypeptide or a modified polypeptide containing a carboxyl group and an amino group, and the carrier protein reacts, by the amino group therein, with the substituted acridine to form a chemical bond;
the protein to be labelled is a protein, a modified protein, a polypeptide or a modified polypeptide containing an amino group, and the amino group in the protein to be labelled reacts with the carboxyl group in the carrier protein to form a —NH—CO— structure, thereby linking the carrier protein and the protein to be labelled together.

2. The acridine-labelled conjugate according to claim 1, wherein the substituted acridine is acridinium ester, acridinic acid, acridine amide or acridine sulfonamide.

3. The acridine-labelled conjugate according to claim 1, wherein the carrier protein is bovine serum albumin, chicken serum albumin or hemocyanin.

4. The acridine-labelled conjugate according to claim 1, wherein the protein to be labelled is an antigen, a hapten or an antibody.

5. A method for preparing the acridine-labelled conjugate according to claim 1, comprising:
covalently cross-linking and fully reacting a substituted acridine with a carrier protein to obtain a substituted acridine-carrier protein conjugate, wherein the carrier protein is a protein, a modified protein, a polypeptide or a modified polypeptide containing a carboxyl group and an amino group, and the carrier protein reacts, by an amino group therein, with substituted acridine to form a chemical bond;
purifying the substituted acridine-carrier protein conjugate;
activating the carboxyl group in a purified substituted acridine-carrier protein conjugate by using a crosslinking agent; and
cross-linking and fully reacting a carboxyl group-activated substituted acridine-carrier protein conjugate with a protein to be labelled to obtain the acridine-labelled conjugate, wherein the acridine-labelled conjugate comprises a substituted acridine, a carrier protein and the protein to be labelled which are sequentially linked, the protein to be labelled is a protein, a modified protein, a polypeptide or a modified polypeptide containing an amino group, and the amino group in the protein to be labelled reacts with the carboxyl group in carrier protein to form a —NH—CO— structure, thereby linking carrier protein and the protein to be labelled together.

6. The method according to claim 5, wherein a molar ratio of substituted acridine to the carrier protein ranges from 100:1 to 20000:1 in covalently crosslinking substituted acridine with the carrier protein.

7. The method according to claim 6, wherein a molar ratio of the carboxyl group-activated substituted acridine-carrier protein conjugate to the protein to be labelled ranges from 5:1 to 1:5 in cross-linking the carboxyl group-activated substituted acridine-carrier protein conjugate with a protein to be labelled.

8. The method according to claim 5, wherein the crosslinking agent comprises carbodiimide and hydroxysuccinimide in activating the carboxyl group in the purified substituted acridine-carrier protein conjugate by using the crosslinking agent.

9. The method according to claim 8, wherein carbodiimide is at least one selected from dicyclohexylcarbodiimide, 1-(3-dimethylamino propyl)-3-ethylcarbodiimide, and N,N′-diisopropyl carbodiimide, and a molar ratio of carbodiimide to the substituted acridine-carrier protein conjugate ranges from 10:1 to 5000:1;
hydroxysuccinimide is at least one selected from N-hydroxysuccinimide and N-hydroxy sulfo succinimide, and a molar ratio of carbodiimide to hydroxysuccinimide ranges from 5:1 to 1:10.

10. A chemiluminescence kit for binding a protein to be labelled to form the acridine-labelled conjugate according to claim 1, comprising a substituted acridine and a carrier protein;
the carrier protein is a protein, a modified protein, a polypeptide or a modified polypeptide containing a carboxyl group and an amino group, and the carrier protein reacts, by the amino group therein, with the substituted acridine to form a chemical bond;
the protein to be labelled is a protein, a modified protein, a polypeptide or a modified polypeptide containing an amino group, and the amino group in the protein to be labelled reacts with the carboxyl group in the carrier protein to form a —NH—CO— structure, thereby linking the carrier protein and the protein to be labelled together.
